(19) United States
(12) Patent Application Publication
(10) Pub. No.: US 2015/0337337 A1
(43) Pub. Date: Nov. 26, 2015

(54) PHYTASE IN BIOGAS PRODUCTION

(71) Applicant: DSM IP ASSETS B.V., Heerlen (NL)

(72) Inventors: Hugo STREEKSTRA, Echt (NL); Theodorus Marius VERLEUN, Echt (NL)

(21) Appl. No.: 14/410,311

(22) PCT Filed: Jun. 25, 2013
(86) PCT No.: PCT/EP2013/063306

§ 371 Date: Dec. 22, 2014

(30) Foreign Application Priority Data
Jun. 26, 2012 (EP) PCT/EP12/62383
Jun. 26, 2012 (EP) PCT/EP12/62385
Jun. 26, 2012 (EP) PCT/EP12/62386
Oct. 1, 2012 (EP) 12186842.6

(51) Int. Cl. C12P 5/02; C12N 9/16

(52) U.S. Cl. CPC C12P 5/023; C12N 9/16; C12Y 301/03; C12P 2201/00; C12P 2203/00

(57) ABSTRACT

The invention relates to a process for the production of biogas from organic material comprising: treating said organic material with an enzyme composition comprising a phytase, preferably a phytase and a cellulase and/or hemicellulase, and digesting the enzyme treated organic material to form biogas, and further to the use of an enzyme composition comprising a phytase to increase the digestibility of proteins and polysaccharides by microbes present in a process suitable for the conversion of organic material into biogas; the use of an enzyme composition comprising a phytase to increase the availability of minerals in a process suitable for the conversion of organic material into biogas; and the use of an enzyme composition comprising a phytase to reduce precipitation of salts on hardware such as (metal) surfaces and in lines and pumps in a process suitable for the conversion of organic material into biogas.

### FIELD OF THE INVENTION

The present invention relates to a process to produce biogas.

### BACKGROUND OF THE INVENTION

The production of biogas via anaerobic digestion of organic material is a rapidly growing source of renewable energy. The process is complex; a combined action of several biotechnological processes determines its stability and efficiency and the yield of the biogas produced. An optimal process design is still under active research, done at laboratory and pilot plants. Substrates like grass, manure or sludge can be used as feed for the biogas production due to their high yield potential.

Phytic acid (or phytate) is an abundant compound in raw materials from plant origin. It is mainly found in seeds, where it serves as a source of phosphorus for the germinating plant. Another function is associated with the molecule's ability to bind minerals: the most common form in plant seeds is phytin, which is the Ca,Mg-salt of phytic acid. Thus, the phytin molecule also serves as a source of these minerals. For this purpose, the plant expresses the phytase enzyme, to break down the phytic acid molecule, and thereby release the phosphorus and the minerals. Also many microorganisms express phytases, to benefit from phytic acid encountered in their growth environment.

The molecular properties of phytic acid may cause problems for processing of raw materials from plant origin. Because of the presence of 6 phosphate groups, the phytic acid molecule carries a net negative charge, even at acidic pH, where many biological compounds are positively charged. This leads to the association of phytic acid with many components within the raw materials, such as proteins and metal ions. But phytic acid may also be associated with neutral compounds, such as starch, for instance when linker molecules are involved.

### SUMMARY OF THE INVENTION

The present invention provides an improved conversion of organic material into biogas by means of addition of a phytase enzyme. The present invention further provides a residual material from the biogas which has a lower amount of phytic acid. The present invention also provides a biogas process which has a lower requirement for trace element addition, less trace elements have to be added to obtain optimal conditions. Moreover the present invention provides an enzyme composition comprising phytase and other enzymes, such as proteases, lipases, cellulases, hemicellulases, and/or pectinases for example useful in a biogas process. Preferably the enzyme composition comprises phytase and cellulase. The biogas process may be conducted as a single-stage process, but also as a multi-stage process. In the case of a multi-stage process, the phytase is preferably applied under conditions that are most suitable for the enzyme's action.

### DETAILED DESCRIPTION OF THE INVENTION

In one aspect the invention provides a process for the production of biogas from organic material comprising:
treating said organic material with an enzyme composition comprising a phytase; and
digesting the enzyme treated organic material to form biogas.

In the present text several terms are explained by phrases found in Wikipedia (http://en.wikipedia.org/).

By biogas is meant the gas product produced by the anaerobic digestion or fermentation of biodegradable materials. Biogas comprises primarily methane and carbon dioxide and may have small amounts of hydrogen sulphide, moisture and siloxanes. In special cases hydrogen or higher alcohols are the targeted product.

By organic matter content of the organic material is meant the dry matter content of the organic material minus ash. COD (Chemical Oxygen Demand) test is commonly used to indirectly measure the amount of organic matter content of the organic material, see for example ISO 6060 (1989).

By enzymatic process or incubation is meant a process which makes use of an enzyme such as a hemicellulase and a pectinase, preferably a hemicellulase, a cellulase and a pectinase, or pectinase to produce a useful product. In the context of the invention, “treating the organic material with an enzyme composition comprising a phytase” is also referred to as “the enzyme treatment”.

By a biogas process or biogas production process is meant the anaerobic digestion or fermentation of biodegradable materials such as biomass, manure, green waste, plant material, and crops. Biogas comprises primarily methane (CH₄) and carbon dioxide (CO₂) and may have small amounts of hydrogen sulphide (H₂S), moisture and siloxanes. The biogas process, more particularly the enzyme digestion of the enzyme-treated organic material to form biogas is carried out in a biogas reactor or biogas-generating reactor. Accordingly, in the context of the invention “digesting the enzyme treated organic material to form biogas” is also referred to as “the biogas reactor” or “the biogas reaction”. The pH of the biogas reactor will in general be between pH of 3 and 8, preferably between pH of 6 and 8. Generally no measures have to be taken to control the pH as the system is capable to maintain this pH itself. In case the substrate of the biogas reactor is outside this pH range, so for example at pH of 5 or lower, or at pH of 9 and higher, the pH of this substrate is preferably neutralized to for example between 6 and 8.

The digesting step can be any type of digestor, and may e.g. be a one-stage or a two-stage digester.

By organic material is meant material that directly or indirectly originates from plant material or from animal material. Typically it is plant-derived polymers. However, manure will also contain organic material from animal origin. Suitable organic material is for example a substrate or feedstock like an energy crop such as poplar, corn, grass, for example switch grass, farm waste like manure or agricultural waste. Also mixes of several organic materials can be used in the process of the invention.

The inventors have noted several problems in biogas production associated to the choice of organic material. Organic material rich in phytic acid, such as cereals, legumes, olives, fruits, nuts and waste streams thereof, tend to bind essential minerals, resulting in reduced availability (effective concentration) of these minerals and a reduced growth or sustainability of said microorganisms. This, in turn, may result for example in reduced production of biogas, reduced conversion of organic material, premature “stoppage” of the biogas process, formation of unwanted side products, etc. Adding phytase may be solve these problems, particularly when using organic material rich in phytic acid.

The inventors have also found that the issues mentioned above not only depend on the type of organic material, but may also vary from batch to batch, particularly the batch-to-batch variation in phytic acid content. It is believed that this is—at least partially—due to the variation in availability of minerals which are required by the microorganism such as acetogens or methanogens to convert the organic material into biogas. In order to overcome this batch-to-batch variation, minerals are often added to the fermentation, for example in the form of a mineral cocktail; this is also referred to as trace elements.

Adding phytase may reduce this batch-to-batch dependency variation and may advantageously give a more robust biogas process.

In the art, to overcome the reduced availability of minerals, biogas producers almost always add minerals to the process. This is a costly matter and the (often toxic) minerals may end up in the biogas reactor or in the environment. Adding phytase may release the minerals that are bound to the phytic acid, and may advantageously avoid adding minerals to the biogas process.

Biogas production using organic material low in phytic acid and/or rich in minerals may not require the addition of such trace elements. However, because biogas processes are very time-consuming (also lab trials), in practice biogas producers tend not take any risk. Instead of testing the requirement of addition of minerals for any given organic material in order to determine the amount of minerals to be added, they tend to always add minerals, whether or not this would be necessary. This has the disadvantage that it is cost-inefficient—trace elements are added in cases where this would not be required for good biogas production. Secondly, adding trace elements means that the biogas plant is enriched with minerals that are potentially poisonous. Thirdly, adding minerals to organic material that by itself already contains sufficient minerals could result in mineral concentrations that are toxic to the (anaerobic) microorganisms.

Adding a phytase is also advantageous when using organic material of which the phytic acid level is unknown, such as manure (from cows, pigs, chicken, horse, or other farm animals), silage (like from corn and/or grass), grass- and other plant-derived raw materials in general, such as brewer's spent grain, distiller's spent grain, distiller's dried grain, sugar beet pulp, corn steep (solids). Such organic materials, particularly organic material from cereal origin, may be not only rich in phytic acid, but are also usually poor in minerals.

Adding phytase may advantageously avoid the need to pre-test the organic material in a (lab trial) biogas process.

Adding phytase may yield consistently good conversion of organic material, whether or not the organic material is rich or low in phytic acid, or rich or low in minerals.

It follows that the effect of adding phytase to a biogas process (e.g. on biogas production) is most pronounced when using organic material which is rich in phytic acid and/or low in minerals. When using organic material which is low in phytic acid and/or rich in mineral, the effect of adding phytase may be less apparent. When minerals are added, the effect of adding phytase may be especially difficult to demonstrate, particularly when the organic material itself is low in phytic acid and/or rich in minerals.

However, even if the effect of adding phytase to a biogas process may be greater with one batch/type of organic material than with another batch/type, the overall (average) effect of adding phytase is still beneficial.

In an embodiment the process comprises, prior to the enzyme treatment:
treating said organic material to reduce the number of viable microorganisms.

If the biogas process includes a recycling step, e.g. recycling of liquid from the biogas reactor, anaerobic microorganisms may be present in the recycle stream that can be introduced into, and may produce biogas in the stages prior to the biogas reaction proper, e.g. during enzyme-treatment. Likewise, such biogas-producing microorganisms may also be present in the organic material, particularly in manure. When using such organic material, and/or if recycling of liquid is desired, measures may have to be taken to prevent biogas production in the first phase.

The organic material is preferably heat-treated or pasteurized at a temperature of 65 to 120° C., more preferably at 65 to 95° C. for a suitable time. Pasteurization is a process of heating the organic material to a specific temperature for a definite length of time in a humid environment. For example pasteurization at 72° C. for 30 seconds is sufficient. For example 1 hour at 120° C. gives the same results as 4 hours at 90° C. with respect to the CFU count. In general, high temperatures may result in more protein denaturation as well as occurrence of toxic compounds. In general, if the pasteurization time is longer, the pasteurization temperature can be lower. The water content at pasteurization should be sufficient to enable pasteurization effect. In general the water content will be between 30 and 95 wt %, preferably between 50 and 90 wt %. This process slows microbial growth in the organic material. Pasteurization or heat-treatment is not intended to kill all microorganisms in the organic material. Instead pasteurization or heat-treatment aims to reduce the number of viable microorganisms so they are unlikely to substantially produce biogas or other fermentation products like organic acids and alcohols in the first stage (or first step or first phase or enzyme treatment) of the process. In general in the first stage less than 2%, preferably less than 1%, of the total of biogas is formed. After the pasteurization or heat-treatment according to the invention the CFU count is in general lower than 10⁶, preferably less than 10⁵, even more preferably less than 10⁴ and most preferably less than 10³ CFU/ml in the organic material present. In microbiology, colony-forming unit (CFU or cfu) is a measure of viable bacterial or fungal numbers. Unlike direct microscopic counts where all cells, dead and living, are counted, CFU measures viable cells. The pasteurization step also facilitates the use of enzymes or enzyme mixtures directly originating from harvested enzyme production fermentations.

Another way to characterize the efficacy of a treatment that reduces in the number of viable microorganisms is by calculating the logarithm of the number of CFUs of the starting material divided by the number of CFUs of the material after the treatment. The advantage of this method is that—since the killing of micoorganisms is generally assumed to be a first-order reaction—the log reduction of a treatment is largely independent of the actual number of microorganisms present. A sterilization procedure may be required to deliver as much as log 10 reduction (which would kill off as many as 10⁸ microorganisms or more), but in the case of the present invention such high efficacy is not required, or not even desirable. An effective treatment procedure in the present invention would deliver at least a log 1 reduction in the number of CFUs, preferably log 2, even more preferably log 3.

In general, it is beneficial for the process to have the thermal treatment at low or high pH, for example a low pH treatment at pH<4, more preferably at pH<3, even more preferably at pH<2, the low pH treatment is in general done at pH>−1, or for example a high pH treatment at pH>8, more preferably pH>9, even more preferably pH>10. Advantages of thermal treatment at high and low pH are for example solubilization and partial hydrolysis of polymers, such as proteins, carbohydrates, such as starch as hemicellulase, and lipids, but also the reduction of viable cells will be enhanced by extreme pH's, resulting in for example a need of lower temperature and/or less time for the thermal treatment. Additional advantages of high pH treatment are for example improving solid/liquid separation at the end of the thermal and enzyme treatments, improved solubilization of protein and fat, and ammonia stripping for feedstocks having high ammonia content. Chemicals to be used for adjustment of the pH can be for example hydrochloric acid, phosphoric acid, and sulphuric acid for lowering the pH, or for increasing the pH potassium hydroxide and sodium hydroxide.

Preferably, in the process of the invention little or no biogas is formed during the enzyme treatment and the biogas production takes place in the biogas reactor. An advantage of treating said organic material to reduce the number of viable microorganisms is that enzymes used, particularly phytase enzymes are hardly inactivated or consumed by microorganisms present. The low numbers of viable microorganisms present have hardly any effect on the enzymes added and their activity.

The organic material is preferably pasteurized or heat-treated at a temperature of more preferably 65 to 120° C., more preferably 65 to 95° C.

During the enzyme treatment and/or separation step anaerobic or aerobic conditions can be maintained. In general no special measures have to be taken to keep anaerobic conditions.

In another embodiment the process comprises, after the enzyme-treatment (and preferably before the biogas reaction):
subjecting the enzyme treated organic material to a solid—liquid separation and recovering the liquid fraction, whereby said liquid fraction is digested to form biogas.

In the solid—liquid separation step the liquid fraction is separated from the solid fraction of the enzyme treated organic material. Preferably optimal conditions are chosen during the solid—liquid separation such as pH, temperature, addition of flocculants or filter aids etc. All kinds of suitable separation techniques can be used such as decantation, filtration, centrifugation or combinations thereof. Optionally flocculant or filter aid is added before the separation takes place in order to improve the separation. Especially flocculants and filter aids which are biologically degradable such as cellulose are advantageously applied. To prevent loss of soluble digestible material the obtained filter cake or centrifuge sludge may be washed. The wash liquor is combined with the primary obtained filtrate or supernatant. To perform these process steps at the enzyme incubation temperatures will facilitate the separation process. The solid fraction from the solid/liquid separation can be processed or used for example by incineration (combustion), composting or spreading on cultivated areas, or forests. The present process having a temperature treatment step, allows composting or spreading of the solid fraction without a further thermal treatment of the solid fraction which is often required in case of spreading of sludge or other biomass.

The liquid fraction can be introduced to a biogas reactor. Upflow anaerobic filters, UASB, anaerobic packed bed and EGSB reactors are examples of high-rate digesters on industrial scale. Especially UASB and EGSB reactors offer benefits of high-rate digesters when applied at high organic loading rates. The use of liquid and solubilized substrate in the biogas reactor enables a very high loading of the reactor. In general 2 to 70 kg COD/m³/day, preferably at least 10 COD/m³/day and/or less than 50 kg COD/m³/day can be introduced in the biogas reactor. More preferably at least 20 kg COD/m³/day can be introduced in the biogas reactor. Preferably the HRT in the EGSB digester is between 3 to 100 hours, more preferably between 3 and 75 hours, even more preferably between 3 and 60 hours and most preferably between 4 and 25 hours. Preferably the HRT in an IC reactor is between 3 to 100 hours, more preferably between 10 and 80 hours and most preferably between 15 and 60 hours. Preferably the HRT in the UASB digester is between 10 to 100 hours, more preferably between 20 and 80 hours and most preferably between 20 and 50 hours. Preferably the HRT in the CSTR digester is between 1 to 20 days, more preferably between 2 to 15 days and most preferably between 2 to 10 days. In general no recycling of liquid to the first stage (enzyme treatment) will take place. In a CSTR system measures can be taken to keep the biomass in the reactor. Preferably the HRT in the anaerobic membrane bioreactor is between 3 to 12 days, more preferably between 4 and 10 days.

The inventors have noted several problems in incubation or fermentation of organic material such as low digestibility of proteins and polysaccharides by microbes present in the process low availability of minerals (metals) which is often compensated by the addition of extra minerals and precipitation of salts on hardware such as on (metal) surfaces and in lines and pumps. Surprisingly the present invention provides an improved process wherein these problems are at least partly solved.

Thus, the invention has several advantages, such as:
(a) a more robust biogas production process;
(b) less batch-to-batch variation;
(c) less dependency on organic material;
(d) possibility to use mixtures of organic material;
(e) better economics due to less or no adding of minerals;
(f) increased production of biogas;
(g) increased biological availability of phosphorus;
(h) opportunity to extract phosphorus from liquid biogas process fractions like (pre-treated-) silage or digestate;
(i) increased biological availability of minerals such as Ca, Mg, Fe, Co, Zn, etc;
(j) opportunity to extract minerals from liquid biogas process fractions like (pre-treated-) silage or digestate;
(k) reduced requirement to add trace minerals;
(l) better use of naturally available trace minerals for the vitality of fermentation microorganisms;
(m) increased accessibility of carbohydrates for carbohydrases;
(n) increased accessibility of proteases to proteins;
(o) increased availability of (essential) amino acids;
(p) increased health of fermentation microorganisms;
(q) less deposit or salt precipitation build-up on metal surfaces in the process hardware;
(r) less cleaning/shutdown requirements;
(s) longer maximum heat transfer possible over these metal surfaces (like in a heat exchanger);
(t) longer use of maximum flow rate plant efficacy; and/or
(u) less pumping energy required.

In an embodiment the organic material is a mixture comprising two or more organic materials, preferably a mixture comprising a grain and manure, preferably pig manure, more preferably a mixture comprising manure and brewer's spent grain or a mixture comprising manure and corn silage, preferably whole corn silage.

In another embodiment the organic material comprises brewer's spent grain. In yet another embodiment the organic material comprises corn silage, preferably whole corn silage.

Brewer's spent grain (also called spent grain, brewer's grain or draff) is the residual grain which remains after the mashing in the beer brewing process. It mainly consists of carbohydrates and proteins, and is rich in phytic acid.

The inventors have surprisingly found that adding phytase, preferably phytase and hemicellulase, is very suitable for biogas production from a mixture comprising two or more organic materials. Such biogas process may be robust and less sensitive to variations between batches, and less sensitive towards the ratio of the two or more organic materials.

Phytic acid may be present in many raw materials used in a biogas process. However, it is usually not known exactly how much phytic acid is present because raw materials are typically variable and poorly controlled, but also because an unknown—and perhaps variable—amount of phytic acid may have been degraded during preceeding processing steps:
Silage, such as made from corn, rye, wheat, barley, grass, may be prepared from only waste material, but often it also includes the seeds. The silage process may degrade some phytic acid, but not completely.
Manure may come from different sources. For monogastric animals, such as fish, chicken and pigs, phytase is often added to the feed, to allow phytic acid breakdown in the intestinal tract of the animal. However, this conversion is not necessarily complete. In contrast, ruminants, such as cows, goats, camels and sheep, and horses usually get fed without phytase supplementation. The ruminal fermentation may degrade phytic acid, but again, the extent to which this happens will be variable.
Waste material from various sources (plants, food) may contain phytic acid. Especially legumes (such as soy, peas, beans, and lupin) and cereals (such as wheat, corn, rye, barley, and oats) contain high levels of phytic acid in their seeds (beans, grains). And also many secondary waste-streams are cereal-based, deriving from processes such as bread making, beer brewing, alcohol production, sugar production. Well-known waste streams include Brewer's spent grain, distillers spent grain, sugar beet pulp, corn steep (solids).
Nuts, kernels, fruit stones, olive pulp, and waste streams from their processing.

The ability of phytic acid to associate with many components within a biomass-derived raw material suggests that advantages may be gained when the phytic acid is removed. For instance, freeing up protein provides nitrogen and carbon for the biogas culture. Freeing up starch or fibers directly provides substrates for biogas formation. It may also allow other enzymes to act more efficiently, by better exposure of their substrates. This may allow lowering the dosage of fiber-degrading enzymes in the process. Liberating minerals (Fe, Zn, Ca, Mg, Co, Cu, Ni, Mn, Mo, V etc.) may provide nutrients for the biogas-producing community. This is of particular interest because trace elements are often dosed during the biogas fermentation, and this may no longer be necessary.

Another advantage of the present invention is a reduction of the total concentration of heavy metals in the digestate which would lessen their proliferation into the environment during disposal. The phosphate that is liberated could also be used as a nutrient for the microbes, but alternatively it may be retrieved from the liquid fraction. In any way, the improved use of minerals and phosphate—and their lower concentration in the digestate—will make the digestate easier to dispose of by—for instance—ploughing into the fields as fertilizer.

Other potential benefits are associated with the properties of phytic acid—when it is still present—in the liquid streams of the biogas fermentation, like substrate streams, fermentation streams, digestate and waste streams. Phytic acid in liquid streams has a tendency to adhere to (metal) surfaces. And since it has retained its ability to bind to other substances in the waste stream, it acts as a primer for fouling layer formation. This may cause many problems in pumps, linings, heat exchangers, etc.

Therefore, the present invention may provide one or more of the abovementioned advantages by breaking down phytic acid in a biogas process. The person skilled in the art should be aware that during determination of phytic acid levels he should take into account in what form and where the phytic acid is present because of its ability to bind to many substances, and its poor solubility in the presence of many compounds.

Even when one avails of an enzyme that can break down phytic acid, it is not trivial to do so in a biogas process. Available commercial phytases have an acid pH optimum. On the one hand they have been selected for this property, because of their intended application in the animal stomach. But on the other hand it must be realized that phytic acid is in general easier broken down at lower pH values because of the increased solubility of the substrate molecule (phytic acid) at low pH, especially in the presence of metal ions. The pH in a biogas reactor, however, is higher than 7.

Therefore, in an embodiment the enzyme treatment is carried out at a pH of 7 or more, preferably at a pH of 7.2 or more, more preferably at a pH of 7.5 or more.

In an embodiment of the invention the treatment of organic material with an enzyme composition comprising a phytase is carried out in the biogas reactor. The main advantage of this procedure is its easy integration into existing production methods. Surprisingly, phytic acid can be effectively broken down under slightly alkaline (pH>7) conditions.

In another embodiment of the invention, the enzyme treatment is carried at a pH of less than 7. The pH may be less than 6.5, more preferably less than 6, even more preferably less than 5.5. This allows the action of the phytase to be optimized separately from the demands of the methanogenic culture in the biogas reactor. This also allows easy combination of phytase with other enzymes or treatments that may have a positive effect on the methane formation in a subsequent reactor. Next to the advantage of being able to optimize the operating conditions independently, there is also the possible benefit of scaling: to use a smaller reactor (with a higher mass throughput per volume) if such a set-up is considered advantageous.

In another embodiment, the phytase enzyme is applied on its own, without the addition of other enzymes.

In yet another embodiment, the phytase enzyme is applied together—either simultaneously or subsequently—with other enzymes preparations, where the phytase and the other enzyme preparations have independent or combined effects.

In yet another embodiment, the phytase enzyme is applied together—either simultaneously or subsequently—with other enzymes preparations, where the phytase and the other enzyme preparations show synergistic effects. That is the presence of the phytase improves the efficacy of the other enzymes, or vice versa. Particularly, a synergistic effect may be obtained by combining phytase plus hemicellulase, even more preferably when combining phytase plus hemicellulase plus cellulase.

A phytase (myo-inositol hexakisphosphate phosphohydrolase) is any type of phosphatase enzyme that catalyzes the hydrolysis of phytic acid (myo-inositol hexakisphosphate) which is an indigestible, organic form of phosphorus that is found in grains and oil seeds, and releases a usable form of inorganic phosphorus.

In an embodiment the enzyme composition further comprises a hemicellulase and/or a hemicellulase.

Enzymes that hydrolyze hemicellulose are usually referred to as hemicellulase. A hemicellulose is any of several heteropolymers (matrix polysaccharides), such as arabinoxylans, present along with cellulose in almost all plant cell walls. While cellulose is crystalline, strong, and resistant to hydrolysis, hemicellulose has a random, amorphous structure with little strength. It is easily hydrolyzed by dilute acid. However, full enzymatic degradation of hemicellulose, in spite of the existence of many different hemicellulase enzymes is difficult to achieve, due to the presence of recalcitrant structures in the polymers.

Cellulases are enzymes that hydrolyze cellulose (β-1,4-glucan or β D-glucosidic linkages) resulting in the formation of glucose, cellobiose, cellooligosaccharides, and the like.

Cellulases have been traditionally divided into major classes: endoglucanases (“EG”, (E.C. 3.2.1.4), which hydrolyze the beta-1,4-linkages between glucose units) (EC 3.2.1.4) (“EG”), exoglucanases or cellobiohydrolases (“CBH”, (E.C. 3.2.1.91), which hydrolyze cellobiose, a glucose disaccharide, from the reducing and non-reducing ends of cellulose) and β-glucosidases ([β]-D-glucoside glucohydrolase (“BG”, (E.C. 3.2.1.21), which hydrolyze the beta-1,4 glycoside bond of cellobiose to glucose). See e.g. Knowles et al., TIBTECH 5, 255-261, 1987; Shulein, Methods Enzymol., 160, 25, pp. 234-243, 1988. Endoglucanases act mainly on the amorphous parts of the cellulose fibre, whereas cellobiohydrolases are also able to degrade crystalline cellulose (Nevalainen and Penttila, Mycota, 303-319, 1995). Thus, the presence of a cellobiohydrolase in a cellulase system is required for efficient solubilization of crystalline cellulose (Suurnakki, et al. Cellulose 7:189-209, 2000). β-glucosidase acts to liberate D-glucose units from cellobiose, cello-oligosaccharides, and other glucosides (Freer, J. Biol. Chem. vol. 268, no. 13, pp. 9337-9342, 1993).

Glycoside hydrolase family 61 (GH61 or sometimes referred to EGIV) proteins are proteins which enhance the action of cellulases on lignocellulose substrates. GH61 was originally classified as endogluconase based on measurement of very weak endo-1,4-β-d-glucanase activity in one family member. The term “GH61” as used herein, is to be understood as a family of enzymes, which share common conserved sequence portions and foldings to be classified in family 61 of the well-established CAZY GH classification system (http://www.cazy.org/GH61.html).

Herein glycoside hydrolase family 61 is used as being member of the family of glycoside hydrolases EC 3.2.1, which is used herein as being part of the cellulases.

The enzyme composition comprising a hemicellulase and a phytase preferably comprises a hemicellulase to phytase ratio of 1:100 to 100:1 (expressed as hemicellulase protein:phytase protein by weight), more preferably comprises a hemicellulase to phytase ratio of 1:10 to 10:1.

The enzyme composition comprising a hemicellulase, a cellulase and a phytase, preferably comprises a hemicellulase and cellulase to phytase ratio of 1:100 to 100:1 (expressed as hemicellulase and cellulase protein phytase protein by weight), more preferably comprises a hemicellulase and cellulase to phytase ratio of 1:10 to 10:1.

According to another aspect, the invention provides a use of an enzyme composition comprising a phytase preferably a hemicellulase and/or a cellulase and a phytase, to increase the digestibility of proteins and polysaccharides by microbes present in a process suitable for conversion of organic material into biogas, to increase the availability of minerals (metals) in a process suitable for the conversion of organic material into biogas, or to reduce precipitation of salts on hardware such as (metal) surfaces and in lines and pumps in a process suitable for the conversion of organic material into biogas.

According to another aspect of the invention an enzyme composition is provided which is useful in the process of the invention.

### Methods and Materials

MethaPlus® L 100 (mixture obtained from DSM, The Netherlands, containing cellulase and hemicellulase).

Phytase 5000 L (phytase preparation obtained from DSM, The Netherlands).

### Example 1

### Laboratory Trial of Phytase, Hemicellulase and Cellulase for Biogas Production

This trial was performed batch-wise in 12 test bottles with 500 ml volume, for 21 days at 39° C. The tests were made in a triplicate (3 bottles for each set of conditions).

The substrate was a mixture of maize silage (60% of organic dry matter of the total substrate mix), cow manure (30% of organic dry matter of the total substrate mix), and wheat grain (10% of organic dry matter of the total substrate mix). Previous to the test, the maize silage was dried (60° C.) and milled to a particle size of 0.5 mm. The wheat grain was milled to the same particle size.

Two enzyme preparations were used: MethaPlus L 100 and Phytase 5000 L.

The following experimental variations were applied (see Table 1)

TABLE 1

| | MethaPlus ® L 100 | Phytase 5000 L |
|---|---|---|
| Control | 0 | 0 |
| MethaPlus ® | 2500 ppm | 0 |
| Phytase | 0 | 5000 ppm |
| MethaPlus ® + Phytase | 2500 ppm | 5000 ppm |

The fermentations (preparations above) were inoculated with sludge from a biogas plant running on the same substrate mixture as used for the test (loading rate: 2.6 kg organic DM/m³×d, Hydraulic Retention Time approx. 65 days). The inoculum was sieved and 400 g inoculum was applied per bottle. A t=0 sample, containing the same inoculum/substrate mixture ratio as the fermentation bottles, was deep-frozen right before the start of the test. Also before the start of the test, the bottles were purged with N2 gas.

After the end of the test, the triplicates were pooled into one sample. All samples (including the thawed t=0 sample) were centrifuged at 1800 rpm, the pellets were washed twice with distilled water, and the washing water was added to the supernatant fractions, which were thereby diluted 2.5-fold. The dry matter of all samples was determined (DIN EN 14346). The original sample, the supernatant and the whole pellet were homogenized by ultrasonic treatment. After homogenization, ca. 10 g homogenized material was microwave-solubilized (3 ml 65% nitric acid; 2 ml 30% hydrogen peroxide; 1000 W microwave).

Then the solubilized liquid was filled up to 25 ml with distilled water, and the mineral constituents were determined via atomic spectroscopy (EN ISO 11885). The following mineral nutrients were determined:

Macro elements: Mg, Ca, P; and trace elements: Fe, Mo, Ni, Mn, Zn, Cu.

The methane production was measured. It was noticed that all enzyme preparations gave an increase in methane production of about 6% compared to the control without enzyme. Also, in the earlier phase of the fermentation there was an extra stimulation of methane production when the enzymes (Methaplus and Phytase) were combined, indicating a positive effect on the activity of the microbial community. This effect was not apparent at the end of the fermentation, indicating that in all fermentations where enzymes had been dosed, a similar extra amount of raw material had been made available for conversion into methane compared to the control without added enzymes.

The soluble and insoluble phosphate were analyzed. It was noticed that the use of the phytase enzyme led to a lowering of phosphate in the insoluble fraction, and an increase in the soluble fraction.

TABLE 2

| | Insoluble P (g/kg DM) | Soluble P (g/kg DM) |
|---|---|---|
| Control | 20.2 | 4.1 |
| MethaPlus ® | 20.0 | 4.2 |
| Phytase | 18.8 | 4.3 |
| MethaPlus ® + Phytase | 16.6 | 4.7 |

Surprisingly, although the cellulase mix Methaplus® only showed a marginal effect on its own, combining the cellulase mix with the Phytase led to a synergetic release of phosphate compared to the phytase alone.

Essentially the same effect was found for Ca, Mg, Fe, Cu, Zn, and Mn. For all these minerals, the soluble concentration was increased by addition of the phytase, especially in combination with the cellulase. For Mo and Ni the concentrations in the supernatant were below the detection limit in all the samples, but here it was also found that the insoluble fraction was decreased by the use of the enzymes.

### Example 2

A large scale biogas test is done using brewer's spent grain (70% of organic dry matter of the total substrate mix). Previous to the test, the brewers spent grain is dried (60° C.) and milled to a particle size of 0.5 mm.

To the brewer's spent grain is added a mineral mixture (XX ppm), MethaPlus L 100 (2500 ppm) and/or Phytase 5000 L (5000 ppm).

The volume of the fermenter is 1254 m³, and the substrate is fed at a rate of 2.5 kg Organic Dry Matter per m³ per day. The enzyme is dosed once per day, at a constant dosage level relative to the substrate feed rate. Due to wash-in and wash-out of the enzyme in the fermenter, the dosage levels indicated in the Table lead to the final equilibrium enzyme concentrations indicated.

The production of biogas is monitored daily, and the amount of biogas is expressed as:

+, production of biogas

++, increased production of biogas

The experiment is repeated 3 times with different brewer's spent grain batches, and the robustness is determined:

−, considerable variation in biogas production between batches

+/−, little variation in biogas production between batches

+, no detectable variation in biogas production between batches

Results are as indicated in Table 3.

TABLE 3

| | | | | |
|---|---|---|---|---|
| Methaplus | Yes | Yes | Yes | Yes |
| Mineral cocktail | No | Yes | No | Yes |
| Phytase | No | No | Yes | Yes |
| Biogas production | + | ++ | ++ | ++ |
| Robustness | − | +/− | + | + |

Similar results are obtained when using a mixture of pig mixture and brewer's spent grain (pig manure:brewer's spent grain=30:70 based on total weight of the mixture); and when using a mixture of pig mixture and whole corn silage (pig manure:whole corn silage=30:70 based on total weight of the mixture).

1. A process for the production of biogas from organic material comprising:
treating said organic material with an enzyme composition comprising a phytase; and
digesting the enzyme treated organic material to form biogas.

2. Process according to claim 1 further comprising, prior to the enzyme treatment:
treating said organic material to reduce the number of viable microorganisms.

3. Process according to claim 1 further comprising, after enzyme treatment:
subjecting the enzyme treated organic material to a solid—liquid separation and recovering the liquid fraction, whereby said liquid fraction is digested to form biogas.

4. Process according to claim 1 wherein the enzyme composition further comprises a hemicellulase and/or a cellulase.

5. Process according to claim 1 wherein the organic material comprises brewer's spent grain.

6. Process according to claim 1 wherein the organic material comprises whole corn silage.

7. Process according to claim 1 wherein the organic material is a mixture comprising two or more organic materials.

8. Process according to claim 1 wherein the organic material is a mixture comprising a grain and manure.

9. Process according to claim 1 wherein the organic material is a mixture comprising manure, optionally pig manure and brewer's spent grain.

10. Process according to claim 1 wherein the organic material is a mixture comprising manure, optionally pig manure and corn silage, optionally whole corn silage.

11. Process according to claim 1 wherein the enzyme treatment is carried out at a pH of 7 or more.

12. Process according to claim 1 wherein the enzyme treatment is carried at a pH of less than 7.

13. An enzyme composition comprising a phytase capable of being used to increase digestibility of one or more proteins and/or polysaccharides by microbes present in a process suitable for conversion of organic material into biogas.

14. An enzyme composition comprising a phytase capable of being used to increase availability of one or more minerals in a process suitable for conversion of organic material into biogas.

15. An enzyme composition comprising a phytase capable of being used to reduce precipitation of one or more salts on hardware optionally comprising one or more (metal) surfaces and/or in lines and pumps in a process suitable for conversion of organic material into biogas.
